(19) United States
(12) Patent Application Publication
(10) Pub. No.: US 2006/0093173 A1
(43) Pub. Date: May 4, 2006

(54) Method and signal processor for reducing feedback in an audio system

(76) Inventors: Volkmar Hamacher, Neunkirchen am Brand (DE); Ulrich Kornagel, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/251,457

(22) Filed: Oct. 14, 2005

Related U.S. Application Data
(60) Provisional application No. 60/618,617, filed on Oct. 14, 2004.

(30) Foreign Application Priority Data
Oct. 14, 2004 (DE) 10 2004 050 304.4

(51) Int. Cl. H04R 25/00; A61F 11/06

(52) U.S. Cl. 381/317; 381/71.11

(57) ABSTRACT

Feedback in audio systems and particularly in hearing aids needs to be detected more reliably, so that it can be filtered as appropriate. For this purpose, the invention provides for the output signal from a signal processing section is modified using a modulation unit to produce a modulated output signal. This modulation must be inaudible to the hearing aid wearer. The modulated signal is fed back via a feedback path to the microphone of the hearing aid. A feedback detector detects the signal modulation and accordingly controls an adaptive filter to compensate for the feedback.

### PROVISIONAL APPLICATION DATA

The present application claims the benefit of the filing date of Provisional Application No. 60/618617 filed Oct. 14, 2004.

### BACKGROUND OF THE INVENTION

1. Field of the Invention

The present invention relates to a method for reducing feedback in an audio system by detecting a feedback signal in an input signal and processing the input signal on the basis of the detected feedback signal to produce an output signal. The present invention also relates to an appropriate signal processing apparatus for an audio system, a mobile radio, a headset, an auditorium sound system and particularly a hearing aid or middle ear implant.

2. Description of the Prior Art

Audio feedback, called feedback below, frequently arises, in hearing aids, particularly when they are high-gain devices. This feedback is expressed as severe oscillations at a particular frequency and can be heard as whistling. This whistling is usually very unpleasant both for the hearing aid wearer and for people who are relatively close by. Feedback can arise, for example, when sound that is picked up via the hearing aid's microphone, amplified by a signal amplifier and output via the earphone, gets back to the microphone and is amplified again.

The simplest approach to feedback reduction is to reduce the hearing aid's gain on a permanent basis, so that the loop gain remains below the critical limit value even in adverse situations. A crucial drawback to this approach is that it is no longer possible to achieve the gains required for more severe hearing impairments. Other approaches measure the loop gain during the hearing aid adjustment and reduce the gain specifically in the critical range using “notch filters” (narrowband rejection filters). Since the loop gains can change constantly in everyday life, the benefit is likewise limited.

To reduce feedback dynamically, a series of adaptive algorithms have been proposed that allow an automatic adaptation to the respective feedback situation and effect appropriate measures. These methods can be roughly divided into two classes.

The first class is “compensation algorithms”, which use adaptive filters to estimate the feedback component in the microphone signal and to neutralize it by subtraction and hence do not adversely affect the hearing aid's gain. These compensation methods presuppose uncorrelated, i.e. ideally white, input signals. Tonal input signals, which always have a higher level of time correlation, result in incorrect estimation of the feedback path, which can lead to the tonal input signal itself being subtracted by mistake.

The second class includes algorithms that do not become active until feedback whistling is present. They generally include an arrangement for detecting the feedback whistling which continuously monitors the microphone signal for feedback oscillation. If oscillations typical of feedback are detected, the hearing aid's gain is reduced at the appropriate point until the loop gain drops below the critical limit. The gain reduction can be effected by lowering a frequency channel or by activating a suitable narrowband rejection filter (notch filter), for example. A drawback is that the oscillation detectors cannot in principle distinguish between tonal input signals and feedback whistling. The result is that tonal input signals are thought to be feedback oscillations and are then inadmissibly lowered in level by the reduction mechanism (e.g. notch filter).

In summary, the manner of operation of all of the adaptive feedback reduction methods is adversely affected by input signals that have a tonal character shaped by dominant sinusoidal signal components (e.g. sounds from a triangle, alarm signals). This frequently results in unacceptable tone impairments in the input signal.

The compensation algorithms frequently involve delay elements with a de-correlating effect being introduced into the signal processing chain in order to prevent tonal signal sections with a length that is characteristic of voice signals from being noticeably attacked. However, echo effects and irritations by desynchronized visual and audio information mean that only delays in the millisecond range are acceptable. It is therefore not possible to avoid reducing music signals, for example, which are frequently correlated over a much longer period.

Another countermeasure is to slow down the filter's adaptation such that all relevant tonal ambient signals are not acted on. However, a consequence of this is also that the compensation filter is no longer able to follow rapid changes in the feedback path fast enough, which means that feedback whistling is produced for a certain time and does not disappear again until the feedback path has stabilized and the filter is adapted with sufficient accuracy again.

The negative consequences of incorrect detection by oscillation detectors are countered by the resultant gain lowering being affected only to a limited extent, which means that tonal useful signals (e.g. alarm signals) that have been mistaken for feedback oscillations, for example, continue to remain audible. However, this presents the risk that in a feedback situation the gain is not lowered sufficiently to drop below the critical limit, and hence the feedback whistling is not eliminated.

PCT Application WO 2001/06746 A discloses stepped control for the compensation filter, where the feedback detector operates on the- basis of the principle of bandwidth detection. If the bandwidth detector recognizes a narrow bandwidth for the hearing aid's input signal in the frequency band that is susceptible to feedback whistling, it is assumed that there is feedback whistling. However, it is not possible to distinguish natural, narrowband signals with spectral components in this frequency band, such as music. In addition, the feedback whistling must represent a dominant signal component in order to be recognized.

Also, EP 1 052 881 A2 discloses an oscillation detector for detecting feedback. In this case as well, the feedback whistling needs to be very distinctly pronounced in order to be recognized.

PCT Application WO 2001/95578 A2 describes detection of feedback whistling by estimating the variance in the frequency estimation of the hearing aid's input signal. This method also has the drawbacks cited above.

In addition, DE 199 04 538 C1 proposes the selective attenuation of individual frequency bands. In this case, frequency bands in which there is feedback whistling are subjected to a greater level of attenuation by an added attenuation element than could be expected for useful signals. The intervention in the forward signal path is sometimes audible to the hearing aid wearer and in addition the detection is probably slow, since the bands are ideally examined in succession.

Another method for reducing feedback in audio systems is known from U.S. Pat. No. 6,347,148. In this case, the spectrum of an input signal is estimated and a psychoacoustic model is used to generate a control signal. The control signal is used to actuate a noise source which can be used to produce an inaudible noise signal on the basis of the noise signal. This document also describes the option of impressing short noise signals of a prescribed duration onto the output signal. The noise signals in the input signal are used to reduce feedback signals.

### SUMMARY OF THE INVENTION

An object of the present invention is to improve the reduction of feedback in a hearing aid further.

This object is achieved in accordance with the invention by a method for reducing feedback in an audio system by detecting a feedback signal in an input signal and processing the input signal to produce an output signal on the basis of the detected feedback signal, and also modulation of the output signal, so that the feedback signal is also correspondingly modulated, with the feedback signal being detected from the modulation.

The invention also provides a signal processing apparatus for an audio system having a processing device for producing an output signal from an input signal by taking into account a feedback signal, a modulation device for modulating the output signal, so that feedback results in a correspondingly modulated feedback signal, and a detection device for detecting the modulated feedback signal from its modulation.

The underlying idea is to impress features that the hearing aid wearer cannot perceive onto the output signal from the audio system and particularly from the hearing aid. This makes it possible to use appropriate analysis of the input signal to determine whether the input signal is feedback or a “normal” external input signal (useful signal). Determining the form of the feature in the input signal also allows inferences about corresponding ratios of feedback to useful signal. This can then be used directly to control feedback reduction algorithms.

Advantageously, it is thus possible to determine, in the course of operation and totally inconspicuously or inaudibly, the extent to which a microphone or the hearing aid's microphone is hearing feedback signals, which allows a significant improvement in the control and action of the known feedback reduction algorithms.

Preferably, the input signal is processed using an adaptable filter whose adaptation speed and/or filtering degree is dependent on the quantity of the detected feedback signal. In particular, it is advantageous if the adaptation speed rises in proportion to the quantity of the detected feedback signal. If the feature analysis of the input signal is then negative, for example, i.e. it does not contain a feedback signal, the adaptation speed of the aforementioned compensation filter can be slowed down such that the filter is not adjusted by tonal input signals and these signals are not attacked. If, however, the feature is detected in the input signal, the filtering degree and/or speed of the feedback compensator is set to the value at which feedback is rejected in optimum fashion.

If a feedback signal is detected then at least one notch filter for processing the input signal can be activated.

The output signal can be modulated by amplitude modulation or modulation of the signal envelope. The perceptibility of the modulation decreases very greatly from approximately 6 Hz modulation frequency onward. Corresponding perception thresholds for the depth of modulation on the basis of the modulation frequency and the signal level are known from psychoacoustics.

Alternatively, the output signal can be modulated by reducing the amplitude to zero and hence by inserting signal gaps, for example. Such signal gaps are no longer perceptible at mid levels below approximately 5 ms.

It is also particularly advantageous to modulate the output signal by phase modulation. This approach also has no particular susceptibility with regard to incorrect detection for narrowband signals.

Generally, it is possible to use all types of signal modulation that are inaudible and can be detected again at the input. In each solution variant, a feedback situation can actually be recognized before the feedback whistling becomes dominant in the signal mix.

Feedback can be detected separately in a number of sub-bands. It is thus possible to adjust the gain, but also the reduction of feedback, individually in the individual sub-bands.

A closed loop in the signal processing apparatus can be used for signal modification. In this case, the modulated signal passes through the loop a plurality of times, so that the corresponding signal modification is brought about.

### DESCRIPTION OF THE DRAWINGS

FIG. 1 shows a hearing aid system based on the prior art.

FIG. 2 shows a hearing aid system based on a first embodiment of the present invention.

FIG. 3 shows a hearing aid system based on a second embodiment of the present invention.

FIG. 4 shows a feedback detector with a filter bank.

### DESCRIPTION OF THE PREFERRED EMBODIMENTS

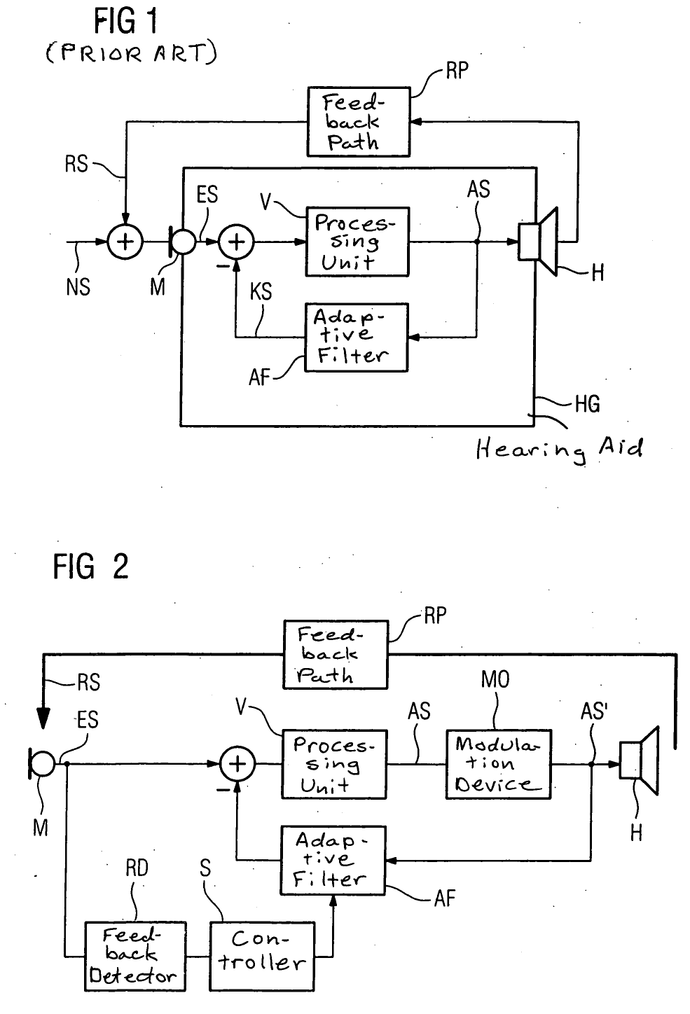

The exemplary embodiments outlined in more detail below are preferred embodiments of the present invention. To provide a better understanding of the invention, the prior art is first explained in more detail with reference to FIG. 1.

FIG. 1 shows a hearing aid HG, whose input is formed by a microphone M. The signal picked up is forwarded as input signal ES to a processing unit V. There, it is processed and possibly amplified. The resultant output signal AS is sent to an earphone H. A feedback path RP is used to feed back the output signal from the earphone H to the microphone M. When the supply is open, there is primarily an audio feedback path. Generally, electromagnetic, electrical, magnetic and other feedback loops are also conceivable, however. The feedback signal RS resulting from the feedback path is added to a useful signal NS, and the summed signal is picked up by the microphone M.

The signal path from the microphone M via the hearing aid processing V, the earphone H, the feedback path RP back to the microphone M is a loop. If the loop gain, i.e. the gain to which a signal is subjected when it passes through this loop, has a value of at least 1.0 at at least one frequency and if the phase condition is satisfied then feedback whistling occurs. Even if the loop gain is just below this limit, audible feedback effects occur, e.g. tone changes.

One successful method for rejecting the feedback effects is digital simulation of the feedback path RP. This feedback path is simulated by an adaptive filter AF to which the output signal from the processing unit V is supplied. An appropriate compensation signal KS coming from the compensating, adaptive filter AF is subtracted from the input signal ES for the microphone M, and the resultant difference signal is supplied to the processing unit V.

There are thus two paths, first the outer feedback path RP and secondly the digital compensation path simulated by means of the adaptive filter AF. The resultant signals on both paths are subtracted from one another at the input to the appliance, as shown in FIG. 1 by the two addition units. Ideally, this cancels the effect of the outer feedback path RP.

An important component in the adaptive algorithm for determining the feedback path is its step size control. This indicates the speed at which the adaptive compensation filter adapts itself to the outer feedback path RP. Since there is no appropriate compromise for a permanently set step size, this needs to be adapted to the respective present audio situation in which the system is present.

In principle, a large step size is desirable for rapid adaptation of the adaptive compensation filter AF to the outer feedback path RP. A drawback of a large step size, however, is the production of perceptible signal artifacts.

If a feedback situation is not present, the step size should be extremely small. In this context, a feedback situation is denoted as that situation in which the loop gain is just below 1 or is greater than or equal to 1 and the phase condition is satisfied at at least one frequency. If a feedback situation occurs, however, the step size should be or become large. This ensures that the algorithm adapts the adaptive compensation filter AF only when the filter's characteristic differs significantly from the characteristic of the feedback path RP, i.e. when re-adaptation is required. For this purpose, a feedback detector is provided.

To be able to detect feedback reliably, the invention provides a modulation device MO which is connected between the processing unit V and the earphone H, as shown in FIG. 2. This device modulates the output signal AS to produce a modulated output signal AS′. The modulation of the output signal AS is not perceptible. In a feedback situation, a significant component of the sound signal which is output by the earphone H gets back to the microphone M and is picked up by the appliance together with the ambient signal.

FIG. 2 indicates that the feedback path RP can basically be in any form. That is to say that it is not necessary to have an audio feedback signal RS, as indicated in FIG. 1, which is added to an audio useful signal NS before the microphone M. Rather, the feedback into the microphone M may also be effected by means of structure-borne noise or electromagnetic interference, for example.

The input signal ES for the microphone M is analyzed by a feedback detector RD. This allows the feedback signal RS to be detected on -the basis of its modulation. A downstream controller S actuates the adaptive compensation filter AF in line with the detection result from the feedback detector RD. This changes the adaptation speed of the adaptive filter AF, for example.

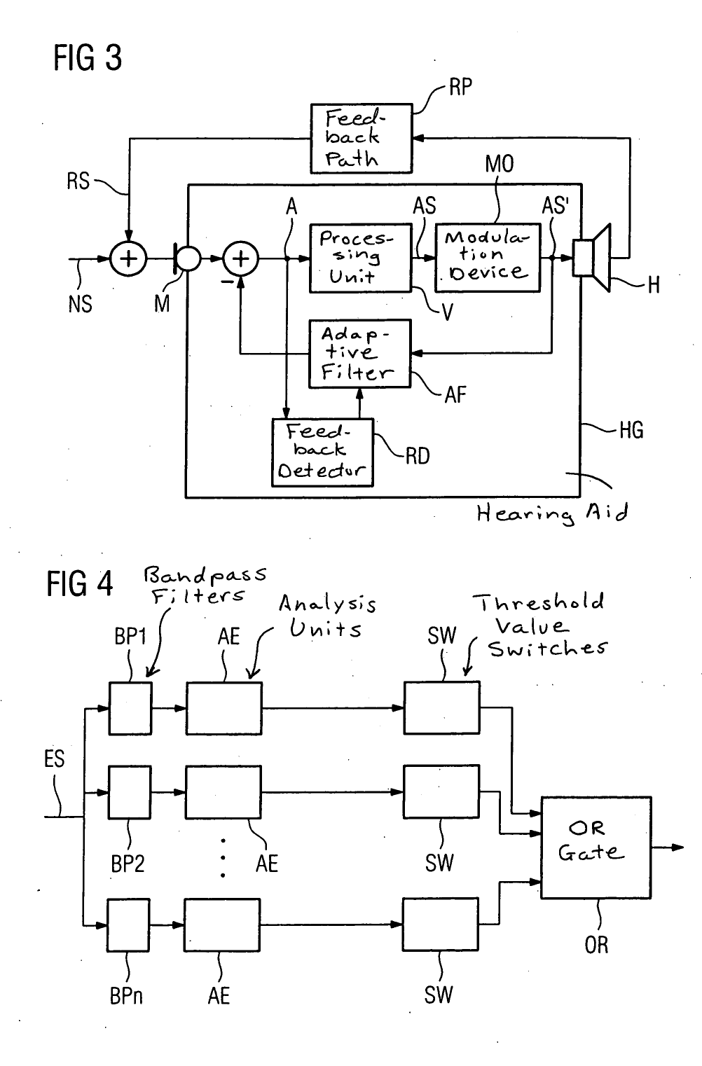

The exemplary embodiment in FIG. 3 essentially corresponds to that in FIG. 2. In this case, the feedback path is of purely audio nature as in the example in FIG. 1, which means that the feedback signal is added to the useful signal before the microphone M.

Another difference from the circuit in FIG. 2 is that the signal for the feedback detector RD is tapped off not directly after the microphone M but rather after subtracting the compensation signal from the adaptive filter AF at point A. The level of signal modulation produced at point A is a depiction of the difference between the action of the feedback path RP and the action of the adaptive compensation filter AF. However, there is no fundamental difference from the embodiment shown in FIG. 2, in which the signal to be analyzed is tapped off directly after the microphone M.

In addition, FIG. 3 indicates that a step size controller can be incorporated into the feedback detector RD, which means that it is possible to dispense with a separate control chip. The other components of the exemplary embodiment in FIG. 3 correspond to those of the exemplary embodiment in FIG. 2. In this regard, reference is thus made to the description relating to FIG. 2.

In the exemplary embodiment shown in FIG. 3, the phase of the output signal AS is modulated, since the human ear is largely insensitive toward phase changes. In a specific example, the phase of the output signal AS is linearly rotated forward and backward between two phase values at a particular frequency, in this case called the modulation frequency f_mod. By way of example, the phase values are □ and □+□/2, where n is any fixed phase. In the feedback situation, a detectable treble component at a frequency of f_mod develops in the signal loop.

The treble component can be detected using a frequency demodulator in the feedback detector RD. In this case, it is beneficial to design the feedback detector RD to have a filter bank, as shown in FIG. 4, which splits the input signal ES into sub-bands using a number of bandpass filters BP1, BP2, . . . , BPn, for example. Downstream of each bandpass filter there is respectively arranged an analysis unit AE and a threshold value switch SW. The output signals from the signal paths for each sub-band are optionally supplied to an OR gate OR. The respective analysis units AE and threshold value switches SW may have the same design as one another. Hence, in this example, the analysis in each sub-band path takes place in the same way. If the analysis result in a band exceeds a certain threshold, the associated threshold value switch SW responds, i.e. a feedback situation is recognized for this band.

This information can be used for an adaptive compensation filter AF adapting in sub-bands for the purpose of step size control. If an adaptive filter AF is used in the whole band, on the other hand, the results of the sub-band detection operations need to be combined into a whole-band detection statement using a logic OR function. Even the special instance in which the whole band is analyzed as one, with n=1, results in an operable system. However, the error detection rate is lower for a larger n, e.g. n=16.

The step size control of the adaptive filter AF can also be effected in more differentiated fashion besides the simple threshold value decision as shown in FIG. 4, where only the presence or absence of feedback is detected. As an example, the step size can be ascertained by virtue of proportional recalculation of the estimated level of the signal modulation at point A. This may also be done using a sub-band approach again. The greater the signal modification recognized, the higher the need for re-adaptation would then be, i.e. the higher the necessary step size would need to be selected. The step size can thus be continually adapted to the signal modulation. In the case of a pure threshold value decision, the step size is, by contrast, stepped up for a certain prescribed time or for the time frame in which feedback is detected. Otherwise, it assumes a small value.

In another embodiment, the phase is not modulated sinusoidally, but rather is changed generally on the basis of a particular profile, e.g. is linearly rotated in one direction (forward or backward). In a feedback situation, a chirp characteristic is then produced for this example in the closed signal loop. To detect the feedback situation, it would then be necessary to use a chirp detector.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for reducing feedback in an audio system comprising the steps of:
in an input audio signal exhibiting modulation, detecting a feedback signal in said audio input signal from said modulation;
processing said input audio signal to produce an output signal dependent on the detected feedback signal; and
modulating said output signal with said feedback signal also being correspondingly modulated.

2. A method as claimed in claim 1 comprising processing said input audio signal with an adaptive filter having an adaptation speed directly dependent on a magnitude of the detected feedback signal.

3. A method as claimed in claim 1 comprising processing said input audio signal with an adaptive filter having an filtering degree directly dependent on a magnitude of the detected feedback signal.

4. A method as claimed in claim 1 comprising processing said input audio signal with an adaptive filter having an adaptation speed that increases in proportion to a magnitude of the detected feedback signal.

5. A method as claimed in claim 1 comprising processing said input audio signal with an adaptive filter having an filtering degree that increases in proportion to a magnitude of the detected feedback signal.

6. A method as claimed in claim 1 comprising detecting said feedback signal with at least one notch filter.

7. A method as claimed in claim 1 comprising modulating said output signal and said feedback signal by amplitude modulation.

8. A method as claimed in claim 7 comprising modulating said output signal to reduce an amplitude of said feedback signal to zero by inserting gaps in said output signal.

9. A method as claimed in claim 1 comprising modulating said output signal and said feedback signal by phase modulation.

10. A method as claimed in claim 1 comprising detecting said feedback signal in said input audio signal in a plurality of sub-bands of said input audio signal.

11. A method as claimed in claim 10 comprising processing said input audio signal with a plurality of adaptable filters respectively operating in said plurality of sub-bands.

12. A signal processor for reducing feedback in an audio system comprising the steps of:
a feedback detector supplied with an input audio signal exhibiting modulation, that detects a feedback signal in said audio input signal from said modulation;
a processing unit supplied with said input audio signal and connected to said feedback detector that processes said input audio signal to produce an output signal dependent on the detected feedback signal; and
a modulation device connected to said processing unit that modulates said output signal with said feedback signal also being correspondingly modulated.

13. A signal processor as claimed in claim 12 comprising an adaptive filter, connected between said feedback detector and said processing unit, having an adaptation speed directly dependent on a magnitude of the detected feedback signal.

14. A signal processor as claimed in claim 12-comprising an adaptive filter, connected between said feedback detector and- said processing unit, having a filtering degree directly dependent on a magnitude of the detected feedback signal.

15. A signal processor as claimed in claim 12 comprising an adaptive filter, connected between said feedback detector and said processing unit, having an adaptation speed that increases in proportion to a magnitude of the detected feedback signal.

16. A signal processor as claimed in claim 12 comprising an adaptive filter, connected between said feedback detector and said processing unit, having a filtering degree that increases in proportion to a magnitude of the detected feedback signal.

17. A signal processor as claimed in claim 12 wherein said feedback detector comprises at least one notch filter.

18. A signal processor as claimed in claim 12 wherein said modulation device modulates said output signal and said feedback signal by amplitude modulation.

19. A signal processor as claimed in claim 18 wherein said modulation device modulates said output signal to reduce an amplitude of said feedback signal to zero by inserting gaps in said output signal.

20. A signal processor as claimed in claim 12 wherein said modulation device modulates said output signal and said feedback signal by phase modulation.

21. A signal processor as claimed in claim 12 comprises a plurality of analysis units that respectively detects said feedback signal in said input audio signal in different sub-bands of said input audio signal.
